(19) United States
(12) Patent Application Publication
(10) Pub. No.: US 2022/0233499 A1
(43) Pub. Date: Jul. 28, 2022

(54) MODIFIED PEG-400 - ASCORBIC ACID (mPEG-AA) COMPLEX AND USES THEREOF

(71) Applicant: INDIAN INSTITUTE OF TECHNOLOGY HYDERABAD, Telangana (IN)

(72) Inventors: Aravind Kumar RENGAN, Telangana (IN); Tejaswini APPIDI, Telangana (IN); Aswathi THOMAS, Telangana (IN); Gayathri R, Telangana (IN); Anil JOGDAND, Telangana (IN); V Sushma MUDIGUNDA, Telangana (IN); Kalaivanan S, Telangana (IN); Prabu Sankar G, Telangana (IN)

(21) Appl. No.: 17/403,964

(22) Filed: Aug. 17, 2021

(30) Foreign Application Priority Data
Jan. 28, 2021 (IN) 202141003895

(51) Int. Cl. A61K 31/375; A61K 47/60

(52) U.S. Cl. CPC A61K 31/375; A61K 47/60

(57) ABSTRACT

The present invention provides a modified PEG-400-ascorbic acid (mPEG-AA complex) comprising the reaction product of fluorescent polyethylene glycol 400 (FLPEG-400) and ascorbic acid. The invention also provides a process for the preparation of the mPEG-AA complex comprising the steps of: (a) heating PEG 400 at a temperature of about 80-90° C. for about 0.5-1 h to get FLPEG-400; (b) adding ascorbic acid to the FLPEG 400; and (c) heating the mixture of FLPEG 400 and ascorbic acid to obtain the mPEG-AA complex. The mPEG-AA complex exhibits both antimicrobial and antiviral activities.

### CROSS REFERENCE TO RELATED APPLICATIONS

The present application claims priority from Indian Patent Application No. 202141003895 filed on Jan. 28, 2021, the contents of which are hereby incorporated by reference herein in their entirety.

### FIELD OF INVENTION

The present invention pertains to the field of advanced materials. More particularly, the invention relates to a modified PEG-400 (mPEG-AA complex) having antibacterial, antifungal and antiviral activity.

### BACKGROUND OF THE INVENTION

Polyethylene glycol (PEG) is used for various biomedical applications such as drug delivery, due to the high biocompatibility and enhanced circulation time they offer. There is no existing polymer technology which acts as both antimicrobial and antifungal while being biocompatible. Since the inhibition of one commodity/population leads to the growth of others, it is necessary to have a material which can act as both antimicrobial and antiviral material.

Polyethylene glycol (PEG), a polymer made out of ethylene oxide monomers is having various applications in chemical to biomedical industries. The structure of PEG is commonly expressed as H—(O—CH₂—CH₂)ₙ—OH. PEG is one of the most used polymers used for biomedical applications as drug carriers, for coating various proteins or molecules (PEGylation). The modifications on physical and chemical properties of PEG are usually performed depending on the application.

There is a continuous effort in development of new materials which can be used as a coating agent and has antimicrobial and antiviral activities. Thus, the present invention represents an advancement in the development of new material to solve a long-standing problem of providing materials which can be used as biocompatible coating agents having antimicrobial and antiviral properties.

### Objectives of the Invention

The primary objective of this invention is to develop a modified PEG 400 having antimicrobial and antiviral activity.

### SUMMARY OF THE INVENTION

The present invention provides a modified PEG-400-ascorbic acid (mPEG-AA complex) comprising the reaction product of fluorescent polyethylene glycol 400 (FLPEG 400) and ascorbic acid. The invention also provides a process for the preparation of the mPEG-AA complex comprising the steps of: (a) heating PEG 400 at a temperature of about 80-90° C. for about 0.5-1 h to get FLPEG-400; (b) adding ascorbic acid to the FLPEG 400; and (c) heating the mixture of FLPEG 400 and ascorbic acid to obtain the mPEG-AA complex. The mPEG-AA complex exhibits both antimicrobial and antiviral activities.

### BRIEF DESCRIPTION OF THE DRAWINGS

FIG. 1 depicts the fluorescence of ascorbic acid added FLPEG (mPEG-AA complex), under white light and UV illumination.

FIG. 2 depicts (a) Density Functional Theory (DFT) calculations have been performed on the model system ascorbic acid-hexaethylene glycol at the B3LYP level of theory using the 6-31++G** basis set. (b) The calculated UV-vis absorption spectrum for the ground state conformer of ascorbic acid-hexaethylene glycol calculated using Time Dependent-Density Functional Theory (TD-DFT). (c) The major electron transition observed in ascorbic acid-hexaethylene glycol.

FIG. 3 depicts (A) Fluorescence of Gold nanoclusters & mPEG-AA complex in different ratios and their fluorescence under UV light; (B) TEM image of GNCs & mPEG-AA complex processed at 37° C.

FIG. 4 depicts CIE chromacity diagram of (A) GNC & mPEG-AA complex, (B) Mixture of GNCs and mPEG-AA complex in different ratios (1:3, 1:1, 3:1 corresponds to GNC:m PEG-AA complex).

FIG. 5 depicts the antibacterial effect of mPEG-AA complex on E. coli and S. aureus. (1: control, 2: PEG 400, 3: FLPEG 400 and 4: mPEG-AA complex).

FIG. 6 depicts the effect of mPEG-AA complex on Candida albicans keeping PEG and FLPEG 400 as experimental controls.

FIG. 7 depicts spot assay of mPEG-AA complex treated Candida albicans.

FIG. 8 depicts experimental evaluation for the combined antibacterial and antiviral efficacy study with mPEG-AA complex.

FIG. 9 depicts (A) Anti-viral efficacy study of mPEG-AA complex on bacteriophage lambda (B) Quantification of the dose-response inactivation of bacteriophage.

### DETAILED DESCRIPTION OF THE INVENTION

As those in the art will appreciate, the following detailed description describes certain preferred embodiments of the invention in detail and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only and is not intended to limit the scope of the invention defined by the appended claims.

The present invention discloses a modified PEG-400 (mPEG-AA complex) having antibacterial, antifungal and antiviral activity. Further, the invention contemplates a multidimensional approach in the development of highly efficacious and cost-effective modified PEG-400.

The present invention is characterized by the following advantages:
(a) Affordable: The modified mPEG-AA complex developed is highly inexpensive and have the potential to act as a coating agent.
(b) Safety: The mPEG-AA complex is biocompatible and does not exhibit any undesirable side effects.
(c) Antimicrobial and antiviral agent: The mPEG-AA complex exhibits both antimicrobial and antiviral activities.

Before the compositions and methods of the present disclosure are described in greater detail, it is to be understood that the invention is not limited to particular embodiments and may vary. It is also to be understood that the terminology used herein is for the purpose of describing particular embodiments only, and is not intended to be limiting since the scope of the formulations and processes will be limited only by the appended claims.

Embodiments described herein can be understood more readily by reference to the following detailed description, examples, and drawings. Elements, apparatus, and methods described herein are merely illustrative of the principles of the present invention and are not limited to the specific embodiments presented in the detailed description, examples, and drawings. Numerous modifications and adaptations will be readily apparent to those of skill in the art without departing from the spirit and scope of the invention.

Unless defined otherwise, all technical and scientific terms used herein have the same meaning as commonly understood by one of ordinary skill in the art to which the methods belong. Although any nanoformulations, compositions or methods similar or equivalent to those described herein can also be used in the practice or testing of the embodiments of the present invention, representative illustrative methods and compositions are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

In one embodiment, the disclosure provides a modified fluorescent polyethylene glycol 400 (mPEG-AA complex) comprising the reaction product of fluorescent polyethylene glycol 400 (FLPEG 400) and ascorbic acid.

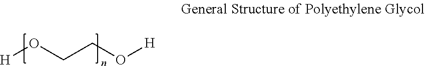

In another embodiment, the mPEG-AA complex exhibits a red shifted fluorescence peak wavelength compared with the peak wavelength of fluorescence emitted by the FLPEG 400 and/or polyethylene glycol 400 (PEG 400).

In another embodiment, fluorescent polyethylene glycol 400 (FLPEG 400) is obtained by heating the PEG 400 at a temperature of about 80-90° C. for about 0.5-1 hr.

In yet another embodiment, ascorbic acid as used for the development of mPEG-AA complex is present as in solid form.

In another embodiment, the PEG 400 is heated at a temperature of about 85-90° C. for about 35-45 minutes for the development of FLPEG-400.

In another embodiment, the concentration of ascorbic acid is at least about 5 mg per mL of FLPEG-400.

In another embodiment, the concentration of ascorbic acid is in a range from 17-50 mg per mL of FLPEG-400.

In another embodiment, the invention provides a method for obtaining a modified polyethylene glycol 400 (mPEG-AA complex) by the steps of:
(a) heating PEG 400 at a temperature of about 80-90° C. for about 0.5-1 hr to get FLPEG 400;
(b) adding ascorbic acid to the FLPEG 400; and
(c) heating under constant stirring of the mixture; FLPEG 400 and ascorbic acid to obtain the mPEG-AA complex.

In another embodiment, in step (a) of the method, PEG 400 is heated at a temperature of about 85-90° C. for about 35-45 minutes.

In another embodiment, in step (b) of the method, the ascorbic acid is added in powder form, wherein the concentration of ascorbic acid to FLPEG is in a range from 5 mg per ml of FLPEG-400 to 50 mg per ml of FLPEG-400.

In another embodiment, ascorbic acid is present at a concentration of at least 5 mg per ml of FLPEG-400.

In another embodiment, in step (c) of the method, the mixture is heated at a temperature of 85-90° C. for about 2-20 minutes.

In another embodiment, the invention provides a composition comprising an effective amount of mPEG-AA complex.

The formulation or composition comprising an effective amount of mPEG-AA complex may further comprise one or more active pharmaceutical ingredient, pharmaceutically acceptable carriers or excipient. The carriers include but are not limited to polymers, sterile aqueous media, solid diluents or fillers, excipients, and various non-toxic organic solvents.

The formulation or composition as disclosed herein can be used as a medicament or as a component in a pharmaceutical composition. Pharmaceutical compositions include tablets, capsules, pills, powders, granules, aqueous and non-aqueous oral solutions and suspensions, sprays, suppositories, gels, pastes, ointments, jellies, lotions, injectable solutions, elixirs, syrups, and parenteral solutions packaged in containers adapted for subdivision into individual doses.

Parenteral formulations include pharmaceutically acceptable aqueous or non-aqueous solutions, dispersion, emulsions, suspensions, and sterile powders for the preparation thereof. Non-limiting examples of carriers include water, ethanol, polyols (such as propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

An effective amount refers to that amount which has the effect of reducing or inhibiting (that is, slowing to some extent, preferably stopping) one or more signs of a medical symptom.

In certain embodiments, composition or formulation containing mPEG-AA complex may be administered in one or more dosage forms.

Those skilled in the art will be able to determine, according to known methods, the appropriate amount, dose or dosage of the composition for administration to a subject taking into account factors such as age, weight, general health, the compositions administered, the route of administration, the nature and advancement of malignancy requiring treatment, and the presence of other medications.

The composition may be administered together or independently of one another by any route known to a person skilled in the art, such as by oral, intravenous, topical, intraperitoneal or nasal route.

In certain embodiments, the composition is administered at a pre-determined daily dosage. This dosage regimen may be adjusted to provide the optimal therapeutic response. For example, the dose may be proportionally reduced or increased as indicated by the exigencies of the therapeutic situation.

The practice of the method of this invention may be accomplished through various administration or dosing regimens. The formulations of the present invention can be administered intermittently, concurrently or sequentially with other prescribed pharmaceutical compositions.

Repetition of the administration or dosing regimens may be conducted as necessary to achieve levels of treatment.

In another embodiment, the mPEG-AA complex may be used as a coating agent in face masks, N95 respirators, elastomeric respirators, powered air-purifying respirators (PAPRs), controlled air-purifying respirators (CAPRs), face shields, personal protection kits and/or other safety accessories and protective packaging covers.

### EXAMPLES

The following examples particularly describe the manner in which the invention is to be performed. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Preparation of Modified PEG-400 (mPEG-AA Complex)

For preparing the modified fluorescent PEG with molecular weight 400 (PEG-400, manufactured by Sigma Aldrich, U.S.A) was heated at a temperature of about 85-90° C. for a period of 40 minutes. This reaction led to the synthesis of FLPEG 400 (PEG-400 with enhanced green fluorescence).

Thereafter, ascorbic acid at a concentration of 5 mg/ml of FLPEG was added to the reaction mixture and continuously stirred at a temperature in the range of 85-90° C. for about 5-7 minutes to obtain modified PEG-400 (mPEG-AA complex).

The mPEG-AA complex exhibits a red shift upon addition of ascorbic acid into the pre-heated samples.

### Example 2: Characterization of the Modified PEG-400 (mPEG-AA Complex)

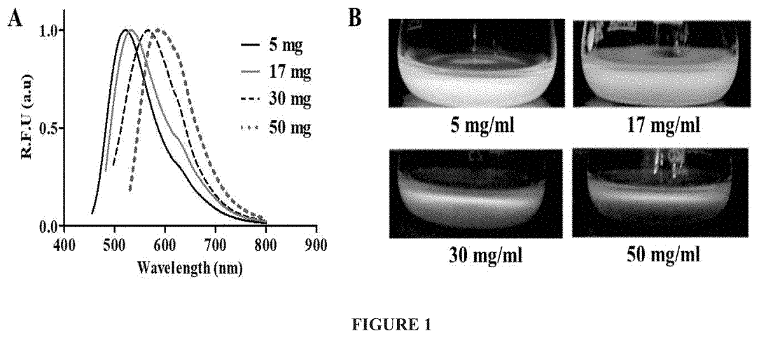

The modified PEG-400 (mPEG-AA complex) as obtained in Example 1 was characterized under white light and UV illumination as depicted in FIG. 1. FIG. 1(a) shows the Ascorbic acid concentration-dependent, fluorescence shifts of mPEG-AA complex. A redshift, in emission spectra, was observed when the concentration of ascorbic acid varied from 5 mg to 50 mg. Under the discussed experimental condition, mPEG-AA complex having the ascorbic acid concentration of 5 mg per ml of FLPEG-400 was found necessary to produce green emission under UV Lamp of 365 nm and was taken for further experiments due to its high intensity as compared to other complexes and flexibility in emission shift within the visible spectrum. FIG. 1(b) shows the digital photographic images of mPEG-AA complex under the UV lamp (365 nm).

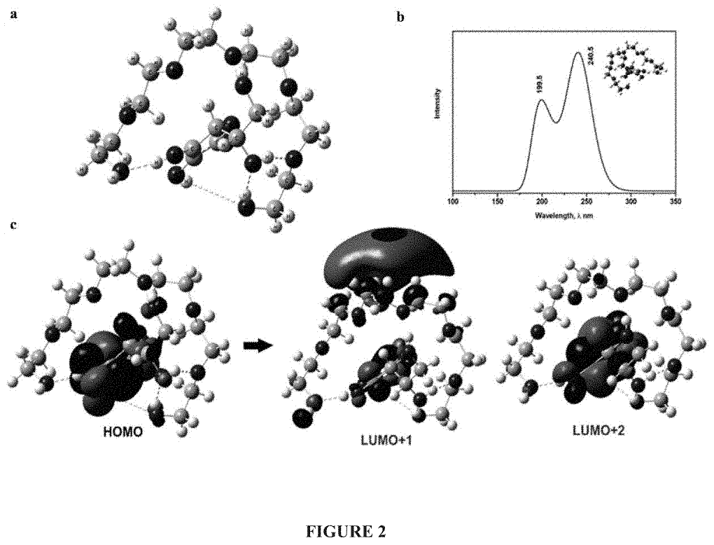

The theoretical study on both ascorbic acid and monoethylene glycol/hexaethylene glycol shows that mPEG-AA complex exist as a single entity through the hydrogen bonding interactions (FIG. 2). Thus, the inventors propose that FLPEG 400 and Ascorbic Acid should coexist in mPEG-AA complex through a strong hydrogen bonding interaction. FIG. 2 depicts (a) Density Functional Theory (DFT) calculations have been performed on the model system ascorbic acid-hexaethylene glycol at the B3LYP level of theory using the 6-31++G** basis set. (b) The calculated UV-vis absorption spectrum for the ground state conformer of ascorbic acid-hexaethylene glycol calculated using Time Dependent-Density Functional Theory (TD-DFT). (c) The major electron transition observed in ascorbic acid-hexaethylene glycol.

### Example 3: Characterization of mPEG-AA Complex Modified with Gold Nanoclusters

PEG 400 was modified with Albumin gold nanoclusters (A-GNCs). Albumin gold nanoclusters (A-GNCs) are inherently red fluorescent. The inventors evaluated the change in the fluorescence with the addition of green fluorescent mPEG-AA complex.

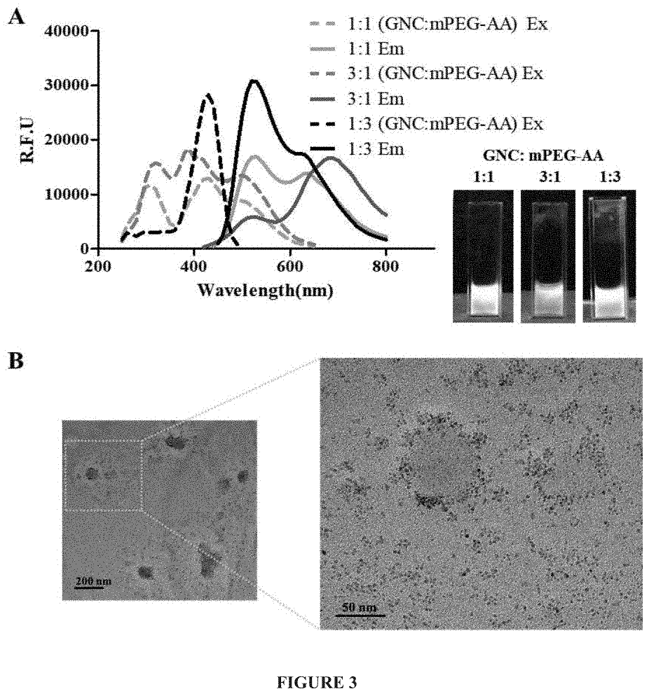

The fluorescence of mixture of A-GNCs and mPEG-AA complex in different ratios and their respective spectra are depicted in FIG. 3.

FIG. 3A shows the fluorescence spectra of A-GNCs & mPEG-AA complex mixed in different ratios and their fluorescence under UV light. A 1:1 mixture of both A-GNCs and mPEG-AA complex show a yellow-colored fluorescence under UV light.

The TEM imaging (FIG. 3B) of A-GNC & mPEG-AA complex (1:1) ratio showed the small dots ranging from 5-7 nm with bigger particles (protein gold nanoclusters:A-GNC).

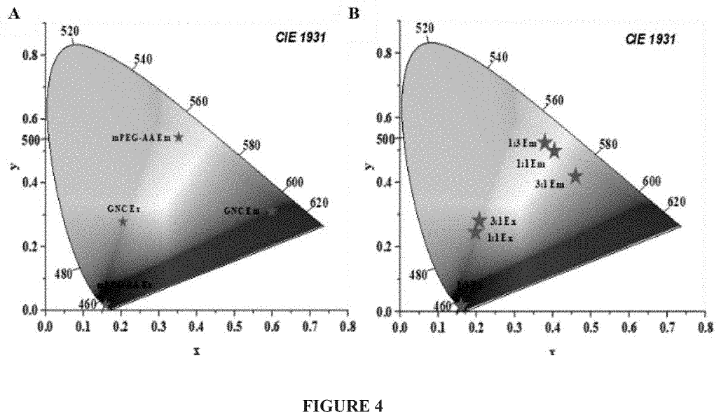

FIG. 4 shows the CIE diagram of GNCs, mPEG-AA complex (FIG. 4A) and their mixtures (FIG. 4B), representing the excitation & emission wavelengths in their associated spectral regions. The emissions of mPEG-AA complex and GNC are blue green and red respectively, while combination of GNCs and mPEG-AA complex in different ratios were yellowish green (1:3), yellowish green (1:1) and orange (3:1).

### Example 4: Antibacterial Effects of mPEG-400

The antibacterial effects of mPEG-AA complex were studied. mPEG-AA complex, modified with ascorbic acid concentration of 5 mg per ml of FLPEG-400 was investigated for antibacterial effect using Escherichia coli and Staphylococcus aureus.

The bacterial culture of Escherichia coli and Staphylococcus aureus was grown till the optical density (O.D.) reached 1.

Thereafter, 200 μl of culture was spread on the LB agar plates separately for each bacterial culture.

A commercially available face-mask was cut into rectangular shape and mPEG-AA complex was coated on the surface of a piece of mask.

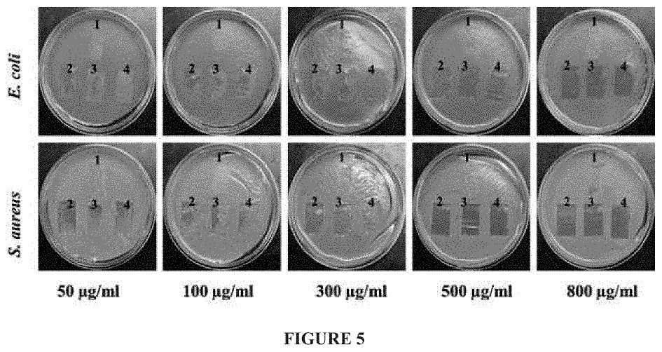

mPEG-AA complex coated mask was placed on the bacteria containing agar plate keeping PEG 400, FLPEG coated and uncoated masks as experimental controls as depicted in FIG. 5. The plates were kept into the incubator at 37° C. for incubation. After 24 hrs, the plates were observed for antibacterial effect of mPEG-AA complex coated and uncoated (control) mask.

Commercially available face-mask material was unable to inhibit the bacterial growth, whereas mPEG-AA complex coated mask was able to inhibit bacterial growth on the agar plate.

Though antibacterial property was visible from a concentration of 50 μg/ml, the mPEG-AA complex was able to inhibit the growth of bacteria at a concentration, starting from 500 μg/ml.

Thus, mPEG-AA complex containing about 5 mg per ml of FLPEG-400 of ascorbic acid was able to inhibit bacterial growth.

FIG. 5 depicts the antibacterial effect of mPEG-AA complex on E. coli and S. aureus (1: control, 2: PEG 400, 3: FLPEG 400 and 4: mPEG-AA complex).

### Example 5: Antifungal Effects of mPEG-AA Complex

The antifungal effects of mPEG-AA complex were studied. Candida albicans was cultured in Sabroud dextrose broth and incubated at 37° C.

The cells were then sub-inoculated in sterile RPMI 1640 media and grown to an optical density (O.D.) of 0.2. The turbidity of the inoculum was adjusted to 0.5 McFarland and diluted in RPMI, corresponding to around 3×10⁵ C.F.U./ml.

The culture was subsequently used for the experiment. 200 μl of culture (triplicates) were plated in the 24-well plate, PEG 400, FLPEG 400 and mPEG-AA complex was added with varying concentrations (50 μg/ml to 300 μg/ml) then incubate the plate at 37° C. for 48 hrs, and viability was evaluated by MTT assay. IC₅₀ value for FLPEG 400 was determined to be 200 μg/ml.

The mPEG-AA complex was found to be a superior antifungal material as compared to FLPEG 400 and PEG 400. The survival of the fungal growth decreases below 50% only when it was treated with 250 μg/ml of FLPEG 400, while it was achieved at a concentration of 100 μg/ml and onwards when treated with mPEG-AA complex.

A spot assay was performed to understand the colony-forming units of Candida albicans. Briefly, 1 mL of five-fold serial dilutions (10, 10⁻¹, 10⁻², 10⁻³ and 10⁻⁴) of Candida albicans inoculum from PEG 400, FLPEG 400 and mPEG-AA complex were treated in a 24-micro well plate and was spotted onto SDA agar plates with proper controls. The growth difference was observed after incubation at 37° C. for 48 hrs.

mPEG-AA complex at a concentration of 200 μg/ml, was observed to possess an antifungal property. The spot assay as well shows the antifungal properties of mPEG-AA complex.

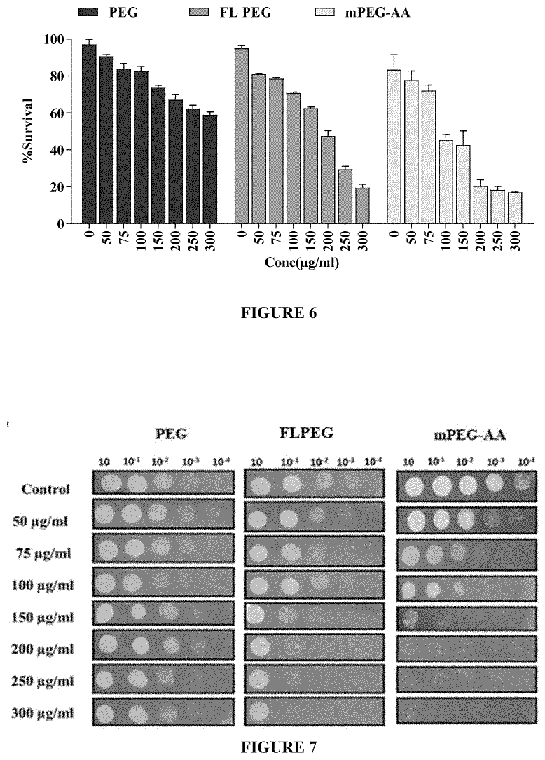

FIG. 6 depicts the effect of mPEG-AA complex on Candida albicans keeping PEG 400 and FLPEG 400 as experimental controls.

FIG. 7 depicts spot assay of mPEG-AA complex treated Candida albicans. FIG. 7 confirms that the growth of fungal colonies can be inhibited with mPEG-AA complex.

### Example 6: Antiviral Effects of mPEG-AA Complex

The antifungal effects of mPEG-AA complex were studied. Varying concentrations of PEG 400, FLPEG 400 and mPEG-AA complex (50 mg and 100 mg) were incubated with bacteriophage lambda viral load of 1*10⁵ pfu/ml in SM buffer.

Post 24 hours of incubation carried out at 37° C., dose-response inactivation was observed in quantitative way of analysis.

Plaque formation assay was performed for the quantitative assessment with which an IC₅₀ value of 200 mg/ml of mPEG-AA complex was observed.

Qualitative analysis with drop-cast method also validates the same, but rather much more pronounced effect was exhibited when incubated with mPEG-AA complex in comparison to the FLPEG 400 of the same concentration.

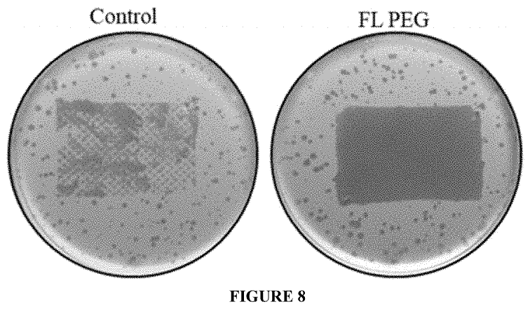

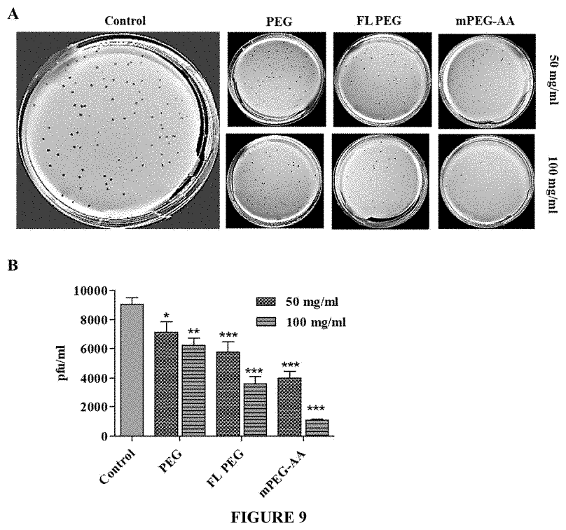

mPEG-AA complex was able to act as an antiviral material with the lowest concentration of 50 mg/ml. It was obtained that a concentration of 100 mg/ml was able to completely inhibit the viral growth in the medium. FIG. 8 depicts experimental evaluation for the combined antibacterial and antiviral efficacy study with mPEG-AA complex.

It was further determined that in the mPEG-AA complex, an ascorbic acid concentration of ˜10 mM inhibits total viral growth.

1. A modified fluorescent polyethylene glycol 400-Ascorbic acid complex (mPEG-AA complex) comprising the reaction product of fluorescent polyethylene glycol 400 (FLPEG 400) and ascorbic acid.

2. The mPEG-AA complex as claimed in claim 1, wherein FLPEG 400 is obtained by heating the PEG 400 at a temperature of about 80-90° C. for about 0.5-1 h.

3. The mPEG-AA complex as claimed in claim 1, wherein the ascorbic acid is present in solid form.

4. The mPEG-AA complex as claimed in claim 2, wherein the PEG 400 is heated at a temperature of about 85-90° C. for about 35-45 minutes.

5. The mPEG-AA complex as claimed in claim 1, wherein concentration of ascorbic acid is at least about 5 mg per ml of FLPEG-400.

6. A modified polyethylene glycol 400 (mPEG-AA complex), obtained by the steps comprising:
a) heating PEG 400 at a temperature of about 80-90° C. for about 0.5-1 h to get FLPEG 400;
b) adding ascorbic acid to the FLPEG 400; and
c) heating under constant stirring of the mixture; FLPEG 400 and ascorbic acid to obtain the mPEG-AA complex.

7. The mPEG-AA complex as claimed in claim 7, wherein, in step a), PEG 400 is heated at a temperature of about 85-90° C. for about 35-45 minutes.

8. The mPEG-AA complex as claimed in claim 7, wherein, in step b), the ascorbic acid is added in powder form and the concentration of ascorbic acid to FLPEG 400 is in a range from 5 mg per ml of FLPEG-400 to 50 mg per ml of FLPEG-400.

9. The mPEG-AA complex as claimed in claim 7, wherein in step c), the mixture is heated at a temperature of 85-90° C. for about 2-20 minutes.

10. A composition comprising an effective amount of mPEG-AA complex as claimed in claim 1.

11. The composition as claimed in claim 11, comprising an effective amount of further comprising one or more active pharmaceutical ingredient, pharmaceutically acceptable carriers or excipient.

12. A process for the preparation of the mPEG-AA, comprising:
a) heating PEG 400 at a temperature of about 60-90° C. for about 0.5-1 h to get FLPEG 400;
b) adding ascorbic acid to the FLPEG 400; and
c) heating the mixture of FLPEG 400 and ascorbic acid to obtain the mPEG-AA complex.

13. The process as claimed in claim 13, wherein FLPEG 400 is obtained by heating the PEG 400 at a temperature of about 80-90° C. for about 0.5-1 h.

14. The process as claimed in claim 13, wherein the ascorbic acid is present in solid form.

15. The process as claimed in claim 13, wherein the PEG 400 is heated at a temperature of about 85-90° C. for about 35-45 minutes.

16. The process as claimed in claim 13, wherein concentration of ascorbic acid is at least about 5 mg per ml of FLPEG-400.

17. A method of treatment comprising administration of a mPEG-AA complex as claimed in claim 1.
